(19) United States
(12) Patent Application Publication
(10) Pub. No.: US 2025/0359764 A1
(43) Pub. Date: Nov. 27, 2025

(54) APPARATUS FOR DETECTING LIMB ISCHEMIA AND METHOD OF USE THEREOF

(71) Applicant: MY01 IP Holdings Inc., Montréal (CA)

(72) Inventor: Olivier Bataille, Beloeil (CA)

(21) Appl. No.: 19/064,617

(22) Filed: Feb. 26, 2025

Related U.S. Application Data
(60) Provisional application No. 63/651,917, filed on May 24, 2024.

(51) Int. Cl. A61B 5/01; A61B 5/00

(52) U.S. Cl. CPC A61B 5/01; A61B 5/6847; A61B 5/7435; A61B 2562/0247

(57) ABSTRACT

An apparatus for detecting limb ischemia in a subject includes a temperature sensor designed to obtain intramuscular temperature readings when implanted into the limb; the apparatus features an introducer for sensor placement and a controller that is configured to: receive the temperature readings from the temperature sensor; and at least one of: display the temperature readings on a display; and analyze the temperature readings to identify a decrease in intramuscular temperature when compared to a reference temperature value, and following the detection, generates a signal or alert indicative of the decrease of intramuscular temperature, whereby the decrease in intramuscular temperature is indicative of a presence of the limb ischemia within the limb; method of use thereof; the apparatus provides a minimally invasive solution for continuous ischemia monitoring, improving early diagnosis, treatment decisions, and patient outcomes.

The present application claims priority from U.S. provisional patent application No. 63/651,917 filed on May 24, 2024, incorporated herein by reference.

### TECHNICAL FIELD

The present disclosure relates to limb ischemia, and more particularly to the diagnosis and management of limb ischemia and/or compartment syndrome.

### BACKGROUND

Limb ischemia occurs when there is a lack of blood flow to a limb. Limb ischemia may be acute (e.g. resulting from an injury or an occlusion from a device placed in the artery or vein) or chronic (where the condition may be referred to as critical limb ischemia), that may result from peripheral vascular disease.

Limb ischemia may lead to, or be related to, serious complications, where limb ischemia can cause severe complications due to reduced blood supply. If left untreated, limb ischemia can lead to intense pain, tissue necrosis, and amputation. Additionally, limb ischemia may contribute to acute compartment syndrome, which can further result in muscle necrosis and loss of limb function.

To mitigate the risk of acute compartment syndrome associated with limb ischemia, distal perfusion catheters (DPC) are sometimes preemptively introduced when limb ischemia is suspected or considered possible. These catheters facilitate procedures such as distal perfusion cannulation to restore circulation if necessary. However, this preventive approach is costly, invasive, and often unnecessary, as not all cases of limb ischemia progress to acute compartment syndrome or result in severe complications. Therefore, an early detection method for diagnosing limb ischemia would be highly beneficial. Such a method could help prevent limb loss, reduce mortality, and mitigate the risk of acute compartment syndrome in cases where a distal perfusion catheter alone may be insufficient. Additionally, such a method would help avoid unnecessary, costly, and invasive procedures, ensuring intervention is only performed when clinically necessary.

### SUMMARY

The present disclosure relates to apparatuses and methods for diagnosing and monitoring limb ischemia and/or compartment syndrome. Limb ischemia can be detected through a measurable decrease in intramuscular temperature, with the extent of temperature change varying based on whether the limb ischemia results from venous or arterial occlusion. Monitoring temperature fluctuations in a limb or muscle where ischemia is diagnosed or suspected provides valuable clinical insights, enabling healthcare professionals to respond swiftly and effectively. It has been shown that a decrease in intramuscular temperature occurs early in ischemia due to reduced perfusion, whereas increased pressure is a hallmark of compartment syndrome. If a temperature change occurs without a corresponding pressure increase, ischemia may be present without compartment syndrome. Conversely, if both temperature decline and pressure elevation are observed, this combination suggests worsening ischemia with a high risk of compartment syndrome.

For instance, if temperature readings indicate no improvement or worsening ischemia after administering treatment (e.g., medication), clinicians can adjust the treatment accordingly. Therefore, continuous temperature monitoring within a suspected ischemic limb or muscle allows healthcare providers to assess the progression of ischemia, determine treatment effectiveness, and/or intervene promptly to prevent further complications. The apparatus of the present disclosure includes a temperature sensor for insertion into a muscle of a subject. Readings from the temperature sensor can be relayed to an external device (e.g. a computing device with a display for viewing). When a drop in intramuscular temperature (e.g. two or three degrees Kelvin) is observed using the apparatus, a presence of limb ischemia may be determined for the subject. Further steps may be taken to resultingly treat the subject (e.g. introduction of a distal perfusion catheter).

In some instances, temperature sensors and pressure sensors of the apparatus may be configured to wirelessly transmit real-time data to a Cloud-based remote server. The remote server may receive the temperature and pressure readings generated respectively by the temperature sensors and the pressure sensors, analyze the temperature readings and pressure readings over time to determine a presence of ischemia, and generate an ischemia alert following the determination. The ischemia alert may be provided to a healthcare provider, where this information can assist in a determination of if a medical intervention is appropriate for the patient from whom the temperature readings and pressure readings are taken.

In some instances, the apparatus, or the remote server in communication with the temperature sensor(s) and pressure sensor(s) of the apparatus, may be adapted to receive temperature readings and pressure readings generated by the temperature sensor(s) and pressure sensor(s) over time, and from the temperature readings and pressure readings, monitor changes in temperature and pressure for the patient following a pharmacotherapy intervention (e.g., thrombolytics, vasodilators) to assess efficacy of the pharmacotherapy intervention. An adjustment to the treatment of the patient may be performed in response to the monitoring of the temperature and pressure of the patient.

In some embodiment, the apparatus may also include a pressure sensor for introduction into a limb (e.g. the muscle) of the subject. The pressure sensor is configured to detect intramuscular pressure in the muscle of the subject. The readings from the pressure sensor may be relayed to an external device (e.g. a computing device, such as the same computing device displaying the readings from the temperature sensor). The readings from the pressure sensor may be used to detect a presence of compartment syndrome with respect to a limb of the subject.

A broad aspect is an apparatus for use in detecting a presence of limb ischemia within a limb of a subject. The apparatus includes a temperature sensor configured to obtain intramuscular temperature readings when implanted into the limb; an introducer adapted to introduce the temperature sensor into the subject; a controller that is configured to: receive the temperature readings from the temperature sensor; and at least one of: display the temperature readings on a display; and analyze the temperature readings to identify a decrease in intramuscular temperature when compared to a reference temperature value, and following the identifying, generate a signal indicative of the decrease of intramuscular temperature, whereby the decrease in intramuscular temperature is indicative of a presence of the limb ischemia within the limb.

In some embodiments, the apparatus may include the display, and the controller may be configured to display the temperature readings on the display.

In some embodiments, the controller may be configured to analyze the temperature readings to identify a decrease in intramuscular temperature when compared to a reference temperature value, and following the identifying, generate a signal indicative of the decrease of intramuscular temperature.

In some embodiments, the signal may be an audio signal emitted through a speaker.

In some embodiments, the signal may be a message appearing on a display.

In some embodiments, the signal may be generated following a determination that the identifying the decrease in intramuscular temperature is occurring over a set period of time according to a period of time value.

In some embodiments, the apparatus may include a pressure sensor that may be configured to generate intramuscular pressure readings of the muscle of the limb once the pressure sensor is introduced into the subject, and wherein the controller may be further configured to receive the pressure readings from the pressure sensor, and at least one of display the pressure readings on the display; and analyze the pressure readings to identify an increase in intramuscular pressure when compared to a reference pressure value, and following the identifying, generate a signal indicative of the increase of intramuscular pressure, whereby the increase in intramuscular pressure is indicative of a presence of compartment syndrome within the limb.

In some embodiments, the temperature sensor may be adapted to transmit the temperature readings to the controller via a wired connection.

In some embodiments, the apparatus may include a user input interface for receiving user input.

Another broad aspect is a use of the apparatus as described herein for detecting limb ischemia in the subject.

Another broad aspect is a use of a temperature sensor to detect limb ischemia in a limb of a subject by identifying a decrease in intramuscular temperature of the limb of a subject when compared to a reference intramuscular temperature value, the decrease in intramuscular temperature indicative of the presence of limb ischemia.

Another broad aspect is a method of detecting a presence of limb ischemia within a limb of a subject. The method includes receiving intramuscular temperature readings of the limb received from a temperature sensor implanted in the limb; analyzing the temperature readings to identify a decrease in intramuscular temperature within the limb when compared to a reference intramuscular temperature value; and outputting information indicative of the decrease in intramuscular temperature, thereby indicative of a presence of limb ischemia within the limb of the subject.

In some embodiments, the method may include introducing a distal perfusion catheter within the limb in order to perform distal perfusion cannula.

In some embodiments, the method may include monitoring intramuscular temperature following the introduction of the distal perfusion catheter to detect an increase in intramuscular temperature.

In some embodiments, the method may include, following an absence of the increase in intramuscular temperature, generating an alert for a risk for compartment syndrome.

In some embodiments, the reference intramuscular temperature value may be an intramuscular temperature value of a healthy limb of the subject.

In some embodiments, the signal may be generated using a signal transducer.

In some embodiments, the method may include implanting the temperature sensor into the limb using an injector.

Another broad aspect is an apparatus for use in detecting, diagnosing or supporting a diagnosis of a presence of one or more of limb ischemia, acute compartment syndrome and peripheral artery disease. The apparatus includes one or more property sensors configured to obtain tissue readings of one or more of temperature and pressure when implanted into the tissue of the subject; an introducer adapted to introduce the one or more property sensors into the subject; a controller that is configured to: receive the tissue readings from the one or more property sensors; and at least one of: display the tissue readings on a display; and analyze the tissue readings to identify a change in one or more of the temperature and pressure when compared respectively to a reference property value or threshold value, and following the identifying, generate a signal indicative of a change in the property or the presence of the one or more of limb ischemia, acute compartment syndrome and peripheral artery disease within the tissue.

Another broad aspect is an apparatus for use in monitoring a state of or detecting a presence of limb ischemia within a limb of a subject. The apparatus includes a temperature sensor configured to obtain intramuscular temperature readings when implanted using an introducer into the limb; a controller that is configured to: receive the temperature readings from the temperature sensor; and at least one of: display the temperature readings on a display; and analyze the temperature readings to identify a change in intramuscular temperature when compared to a reference temperature value, and following the identifying, generate information indicative of the change of intramuscular temperature, whereby the change in intramuscular temperature is indicative of a change in the state of limb ischemia or the presence of limb ischemia.

In some embodiments, the apparatus may include the introducer.

In some embodiments, the apparatus may include the display, and the controller may be configured to display the temperature readings on the display.

In some embodiments, the controller may be configured to analyze the temperature readings to identify a change in intramuscular temperature when compared to a reference temperature value, and following the identifying, generate the information indicative of the change of intramuscular temperature.

In some embodiments, the information may be a signal, and the signal may be generated following a determination that the identifying the change in intramuscular temperature may be occurring over a set period of time according to a period of time value.

In some embodiments, the change in intramuscular temperature may be a decrease in intramuscular temperature for detecting the presence of limb ischemia.

In some embodiments, the information may be a signal that may be an audio signal emitted through a speaker.

In some embodiments, the information may be a signal that is a message appearing on a display.

In some embodiments, the apparatus may include a pressure sensor for insertion into the limb, and the controller may be further configured to receive pressure readings generated by the pressure sensor.

In some embodiments, the controller may be further configured to perform at least one of: display the pressure readings on the display; and analyze the pressure readings to identify a change in intramuscular pressure when compared to a reference pressure value, and following the identifying, generate a signal indicative of the change of intramuscular pressure, whereby the change in intramuscular pressure may provide information on a presence of compartment syndrome within the limb.

In some embodiments, the temperature sensor may be adapted to transmit the temperature readings to the controller via a wired connection.

In some embodiments, the apparatus may include a user input interface for receiving user input.

In some embodiments, the temperature sensor may be a first temperature sensor. The first temperature sensor may include a plurality of temperature sensors including the temperature sensor, and the controller may be further configured to receive temperature readings from the plurality of temperature sensors.

Another broad aspect is a use of the apparatus as described herein for detecting limb ischemia in the subject.

Another broad aspect is a use of a temperature sensor to detect limb ischemia in a limb of a subject by identifying a change in intramuscular temperature of the limb of a subject when compared to a reference intramuscular temperature value, the change in intramuscular temperature indicative of the presence of limb ischemia.

Another broad aspect is a method of detecting a presence of limb ischemia within a limb of a subject. The method includes receiving intramuscular temperature readings of the limb received from a temperature sensor implanted in the limb; analyzing the temperature readings to identify a decrease in intramuscular temperature within the limb when compared to an expected temperature standard; and outputting information indicative of the decrease in intramuscular temperature, that is indicative of a presence or a state of limb ischemia within the limb of the subject.

In some embodiments, the method may include introducing a distal perfusion cannula within the limb.

In some embodiments, the method may include monitoring intramuscular temperature following the introduction of the distal perfusion cannula to detect an increase in intramuscular temperature.

In some embodiments, the method may include following an absence of the increase in intramuscular temperature, generating an alert indicative of a risk of compartment syndrome and low perfusion that may be tied to tissue necrosis or acute coronary syndrome.

In some embodiments, the reference intramuscular temperature value may be an intramuscular temperature value of a healthy limb of the subject.

In some embodiments, the signal may be generated using a signal transducer.

In some embodiments, the method may include implanting the temperature sensor into the limb using an injector.

In some embodiments, the method may include receiving pressure readings of the limb received from a pressure sensor implanted in the limb; analyzing the pressure readings to identify a change in pressure within the limb when compared to a reference value; and outputting information indicative of the change in pressure, which may be indicative of an abnormality within the limb of the subject.

Another broad aspect is an apparatus for use in detecting a presence of, diagnosing or aiding in the diagnosis of one or more of limb ischemia, acute compartment syndrome, peripheral artery disease, or an effect of pharmacotherapy within a tissue of a subject. The apparatus includes a temperature sensor configured to obtain temperature readings within the tissue when implanted into the tissue; a pressure sensor configured to obtain pressure readings within the tissue when implanted into the tissue; and a controller that is configured to: receive the temperature readings from the temperature sensor and the pressure readings from the pressure sensor; and at least one of: display the temperature readings and the pressure readings on a display; and analyze the temperature readings and the pressure readings to identify a change in one or more of temperature and pressure within the tissue when compared respectively to a reference standard, and following the identifying, generate information indicative of the change in the one or more of the temperature and pressure, related to a state or presence of the one or more of limb ischemia, acute compartment syndrome and peripheral artery disease within the tissue.

Another broad aspect is a system for detecting or monitoring a presence of limb ischemia within a limb of a subject. The system includes a processor and memory that includes program code that, when executed by the processor, causes the processor to: receive intramuscular temperature readings of the limb received from a temperature sensor implanted in the limb; analyze the temperature readings to identify a decrease in intramuscular temperature within the limb when compared to a reference intramuscular temperature standard; and output information indicative of the decrease in intramuscular temperature, related to a state of limb ischemia within the limb of the subject.

In some embodiments, the program code, when executed by the processor, may further cause the processor to monitor intramuscular temperature following the introduction of a distal perfusion cannula to detect an increase in intramuscular temperature.

In some embodiments, the program code, when executed by the processor, may further cause the processor to, following an absence of the increase in intramuscular temperature, generate an alert for a risk for compartment syndrome.

In some embodiments, the reference intramuscular temperature value may be an intramuscular temperature value of a healthy limb of the subject.

In some embodiments, the signal may be generated using a signal transducer.

In some embodiments, the program code, when executed by the processor, may further cause the processor to: receive pressure readings of the limb received from a pressure sensor implanted in the limb; analyze the pressure readings to identify a change in pressure within the limb when compared to a reference value; and output information indicative of the change in pressure, thereby indicative of a presence of compartment within the limb of the subject.

In some embodiments, the monitoring may be to determine a change in the state of limb ischemia following an administration of a treatment.

In some embodiments, the analyzing may be performed using an artificial intelligence model that may have been trained using a dataset of historical temperature data associated to a plurality of patients, the dataset of historical temperature data correlating a state of limb ischemia to historical temperature readings over time for each patient.

Another broad aspect is a non-transitory computer-readable medium having stored thereon program instructions for detecting a presence of limb ischemia within a limb of a subject, the program instructions executable by a processing unit for: receiving intramuscular temperature readings of the limb received from a temperature sensor implanted in the limb; analyzing the temperature readings to identify a decrease in intramuscular temperature within the limb when compared to a reference intramuscular temperature value; and outputting information indicative of the decrease in intramuscular temperature, related to a limb ischemia within the limb of the subject.

In some embodiments, the program instructions may be further executable by a processing unit for monitoring intramuscular temperature following an introduction of a distal perfusion catheter to detect an increase in intramuscular temperature.

In some embodiments, the program instructions may be further executable by a processing unit for, following an absence of the increase in intramuscular temperature, generating an alert for a risk for compartment syndrome.

In some embodiments, the reference intramuscular temperature value may be an intramuscular temperature value of a healthy limb of the subject.

In some embodiments, the signal may be generated using a signal transducer.

In some embodiments, the non-transitory computer-readable medium may include implanting the temperature sensor into the limb using an injector.

In some embodiments, the program instructions may be further executable by a processing unit for: receiving pressure readings of the limb received from a pressure sensor implanted in the limb; analyzing the pressure readings to identify a change in pressure within the limb when compared to a reference value; and outputting information indicative of the change in pressure, which may be indicative of a presence of compartment syndrome within the limb of the subject.

Another broad aspect is a method of monitoring a presence of limb ischemia related to a limb of a patient. The method includes monitoring temperature readings taken from within the limb of the patient to detect a change in temperature of the limb that is indicative of a presence or change of state of limb ischemia with respect to the limb of the patient.

In some embodiments, the method may include monitoring pressure readings taken from within the limb of the patient to detect a change in pressure of the limb that may be indicative of a presence or change of state of compartment syndrome with respect to the limb of the patient.

In some embodiments, the monitoring may occur following an administration of a treatment for the limb ischemia.

In some embodiments, the method may include adjusting the treatment in accordance with the detecting that results from the monitoring.

In some embodiments, the monitoring may occur after a diagnosis of limb ischemia, and the monitoring may be to detect a change in temperature that may be indicative of a change of state of the limb ischemia corresponding to a convalescence or a worsening of the limb ischemia.

In some embodiments, the method may include administering a treatment following the detection of limb ischemia resulting from the monitoring.

In some embodiments, the method may include controlling a flow through the distal perfusion cannula based on the intramuscular temperature.

In some embodiments, the artificial intelligence model may be further trained using a dataset of historical pressure data associated to a plurality of patients, the dataset of historical pressure data correlating a state of limb ischemia to historical pressure readings over time for each patient.

### BRIEF DESCRIPTION OF THE DRAWINGS

The invention will be better understood by way of the following detailed description of embodiments of the invention with reference to the appended drawings, in which:

FIG. 1A is a graphical representation of an exemplary apparatus for detecting a presence of limb ischemia;

FIG. 1B is a drawing of an isometric view of an exemplary apparatus for detecting a presence of limb ischemia;

FIG. 2A is a block diagram including an exemplary computing device of an exemplary apparatus for detecting a presence of limb ischemia;

FIG. 2B is a block diagram of an exemplary software architecture for limb ischemia state evaluation;

FIG. 3 is a flowchart diagram of an exemplary method for detecting a presence of limb ischemia in a subject;

FIG. 4 are graphs illustrating a change in intramuscular pressure over time and a change in intramuscular temperature over time for subject 1;

FIG. 5 are graphs illustrating a change in intramuscular pressure over time and a change in intramuscular temperature over time for subject 2;

FIG. 6 are graphs illustrating a change in intramuscular pressure over time and a change in intramuscular temperature over time for subject 3;

FIG. 7 are graphs illustrating a change in intramuscular pressure over time and a change in intramuscular temperature over time for subject 4;

FIG. 8 are graphs illustrating a change in intramuscular pressure over time and a change in intramuscular temperature over time for subject 5;

FIG. 9 are graphs illustrating a change in intramuscular pressure over time and a change in intramuscular temperature over time for subject 6;

FIG. 10 are graphs illustrating a change in intramuscular pressure over time and a change in intramuscular temperature over time for subject 7;

FIG. 11 are graphs illustrating a change in intramuscular pressure over time and a change in intramuscular temperature over time for subject 8;

FIG. 12 are graphs illustrating a change in intramuscular pressure over time and a change in intramuscular temperature over time for subject 9; and

FIG. 13 are graphs illustrating intramuscular temperature and pressure changes over time in the same patient, where one leg was treated with a distal perfusion cannula (DPC) while the other leg served as a control.

### DETAILED DESCRIPTION

It has been discovered that a change (e.g. a decrease) in intramuscular temperature may be an indicator of a presence (a development) of limb ischemia within the limb. For example, a decrease in intramuscular temperature may be an indicator of a presence (or a development) of limb ischemia within the limb. A change in intramuscular temperature, such as an increase or a combination of increases and/or decreases over a period of time forming certain trends, may provide information on a state (e.g. an improvement, a worsening, a development, etc.) of limb ischemia within the tissue of the patient. The present disclosure relates to apparatuses and methods used for detecting a presence of limb ischemia in a subject by monitoring intramuscular temperature of a limb of a subject, and identifying a change (e.g. a decrease, an increase, a certain trend, etc.) in intramuscular temperature that is indicative of a presence of limb ischemia. Moreover, the temperature readings may also be used to detect a change of state in limb ischemia afflicting a limb of the subject. For instance, a fluctuation in temperature within the limb may be indicative of a worsening or improvement of limb ischemia. Corrective action may be taken following the detection of the change in state of limb ischemia, such as changing the treatment regimen, performing surgery, administering a treatment regimen, etc.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

In the present disclosure, by “patient” or “subject”, used interchangeably herein, it is meant a mammal, such as a human. The term “subject” or “patient” should not bring on any limitations as to the sex or age.

The term “treat”, “treating” or “treatment”, as used herein, refers to alleviating or improving the outcome of the subject with regard to a given disease or disorder, which may be quantifiable by improving at least one physical parameter, ailment or biomarker of the subject with the disease or disorder.

The term “reading” or “readings”, as used herein, refers to a representation of data collected from a device which may be a numerical value, a graph, a trendline, etc.

### Exemplary Apparatus for Use in Detecting Limb Ischemia:

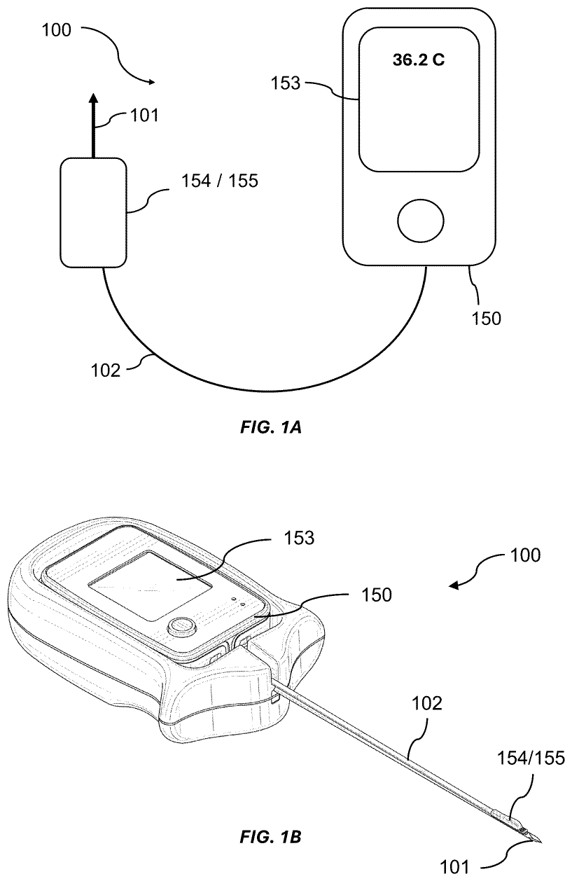

Reference is made to FIG. 1A, illustrating an exemplary apparatus 100 for use in the detecting the presence of limb ischemia in a limb of a subject. FIG. 1B illustrates a further implementation of the apparatus 100 of FIG. 1B.

The apparatus 100 includes a computing device 150 (a controller enclosed within a housing) and a temperature sensor 154. The apparatus 100 may include a pressure sensor 155. The apparatus includes an injector 101 joined to the temperature sensor 154, and when present, to the pressure sensor 155. The apparatus 100 may further include pressure vents for the calibration of the pressure monitor. The calibration may occur after manual initiation or automatically at strategically predetermined times such as, but not limited to, the activation of the apparatus 100, the restarting of the apparatus 100, the passing of a certain time interval, etc. An exemplary injector 101 is illustrated at FIG. 1B.

The apparatus 100 may include a wire or cable 102 connecting the computing device 150 to the temperature sensor 154, and, when present, the pressure sensor 155 (it will be understood that, in some instances, each of the temperature sensor 154 and the pressure sensor 155 may include a cable 102 for connection to the computing device 150).

The computing device 150 may include a display 153 for, e.g., displaying the readings received from the temperature sensor 154 and/or the pressure sensor 155.

The injector 101 is used to pierce the skin of the subject, and for introducing the temperature sensor 154, and, when present, the pressure sensor 155, under the skin of the subject (e.g. into the muscle of the subject). In some instances, the injector 101 is a needle. The injector 101 may allow the skin to be pierced a plurality of times in order for the property sensors (temperature and/or pressure) to collect multiple data values or to achieve optimal data collection. The injector 101 may also remain in the skin for certain duration of time or may be removed once the sensors are introduced. In some embodiments, the injector 101 may be able to provide tactile feedback while piercing tissue.

The temperature sensor 154 is configured for measuring the temperature within the muscle of the subject. The temperature sensor 154 may be one as is known in the art for taking intra-muscular temperature readings.

The pressure sensor 155 is configured for measuring the pressure within a muscle of the subject. The pressure sensor 155 may be one as is known in the art for taking intra-muscular pressure readings. The pressure sensor 155 may be fluid filled or a micro-electromechanical system (MEMS) such as a piezoresistive pressure monitor or capacitive pressure sensor. The pressure sensor 155 may also include a barometric pressure sensor. The barometric pressure sensor may be used to determine differential pressure between the intra-muscular pressure readings and atmospheric pressure. The differential pressure may allow the apparatus to provide more robust readings that withstand changes in altitude.

In some instances, the cable 102 may be absent, where the temperature sensor 154, and, when present, the pressure sensor 155, transmits respective readings to the computing device 150 (wirelessly).

In other embodiments, when the cable 102 is present, the temperature sensor 154, and, when present, the pressure sensor 155, transmit the respective readings to the computing device 150 via the wired connection provided via the cable 102.

In some instances, multiple temperature sensors 154 may be provided, each in communication with a same computing device 150 that receives the temperature readings from the temperature sensors 154. Each temperature sensor 154 may be inserted into a different tissue of the patient, thereby providing temperature readings for monitoring a plurality of tissues or muscles. In some instances, a temperature sensor may be inserted into an abdominal muscle and one or more temperature sensors may be inserted into a limb of interest. The data collected by the temperature sensor inserted into the abdominal muscle may be used as a standard for other temperature sensors inserted into other muscles or limbs of the patient to determine if there is a change in temperature by calculating a difference between the temperature readings generated by the temperature sensor inserted into the abdomen and the temperature readings generated by the temperature sensor(s) into other muscles or limbs of the patient. In another instance, a plurality of temperature sensors may be inserted into the patient, where readings generated by the plurality of temperature sensors may be analyzed to obtain a plurality of data points for statistical analysis of the temperature readings generated from a limb or tissue of interest.

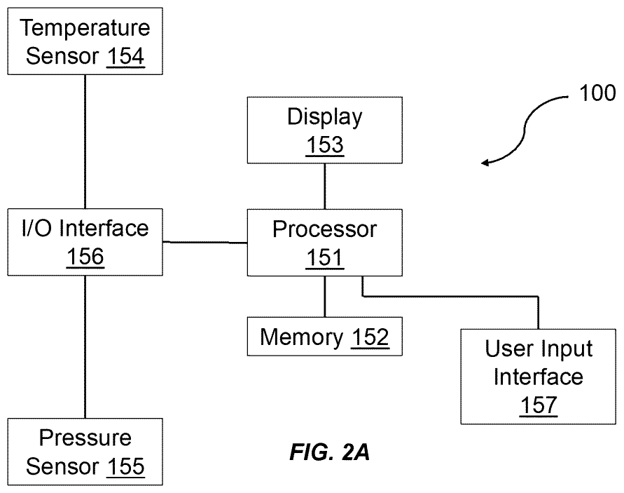

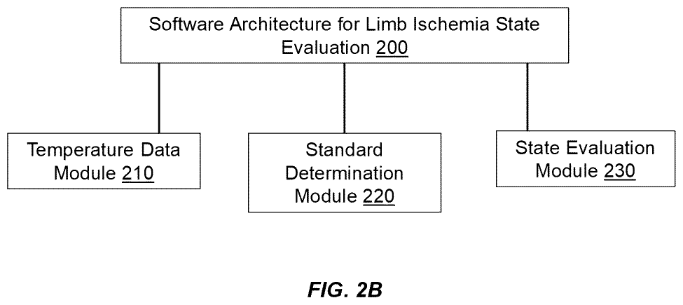

In some instances, multiple pressure sensors 155 may be provided, each in communication with a computing device 150 that receives the pressure readings from the pressure sensors 155. Each pressure sensor 155 may be inserted into a different muscle of the patient, thereby providing pressure readings for monitoring a plurality of muscles. In some instances, a pressure sensor may be inserted into an abdominal muscle and one or more pressure sensors may be inserted into a limb of interest. The data collected by the pressure sensor inserted into the abdominal muscle may be used as a standard for other pressure sensors inserted into other muscles or limbs of the patient to determine if there is a change in pressure by calculating a difference between the pressure readings generated by the pressure sensor inserted into the abdomen and the pressure readings generated by the pressure sensor(s) inserted into other muscles or limbs of the patient. In another instance, a plurality of pressure sensors may be inserted into the patient, where readings generated by the plurality of pressure sensors may be analyzed to obtain a plurality of data points for statistical analysis of the pressure readings generated from a limb or tissue of interest. With reference to FIG. 2A, the computing device 150 includes a processor 151 and memory 152 in communication with the processor 151. The computing device 150 includes an Input/Output (I/O) interface 156. The computing device 150 may include a display 153. The computing device 150 may include a user input interface 157. It will be understood that in some instances, the computing device 150 may include a microprocessor. The microprocessor, or the processor 151 and memory 152, may be referred to herein as a controller. The controller may also be a circuit board, as explained herein.

The computing device 150 may be a dongle with a microprocessor, a portable computer, a smartphone, a laptop, a desktop computer, a tablet, a smartwatch, etc.

The processor 151 may be a general-purpose programmable processor.

The computer readable memory 152 stores program instructions and data used by the processor 151. The computer readable memory 152 may also store temperature readings, pressure readings, temperature standards such as temperature value for detecting a change in temperature readings, etc. The memory 152 may be non-transitory. The computer readable memory 152, though shown as unitary for simplicity in the present example, may comprise multiple memory modules and/or caching. In particular, it may comprise several layers of memory such as a hard drive, external drive (e.g. SD card storage) or the like and a faster and smaller RAM module. The RAM module may store data and/or program code currently being, recently being or soon to be processed by the processor 151 as well as cache data and/or program code from a hard drive. A hard drive may store program code and be accessed to retrieve such code for execution by the processor 151 and may be accessed by the processor 151 to store and access data. The memory 152 may have a recycling architecture for storing, for instance, temperature readings, pressure readings, etc., where older data files are deleted when the memory 152 is full or near being full, or after the older data files have been stored in memory 152 for a certain time.

The I/O interface 156 is in communication with the processor 151. The I/O interface 156 may include a network interface and may be a wired (through cable 102) or wireless interface for establishing a connection with the temperature sensor 154, and when present, the pressure sensor 155, to receive the readings from the temperature sensor 154, and when present, the pressure sensor 155. It will be understood that in some embodiments, when both the temperature sensor 154 and pressure sensor 155 are present, each of the temperature sensor 154 and pressure sensor 155 may transmit readings to a respective I/O interface (not shown) of the computing device 150 through a wired or wireless connection.

The processor 151, the memory 152 and the I/O interface(s) 156 may be linked via bus connections.

The user input interface 157 is a device through which the user may provide input to the computing device 150. A user input interface 157 may be, or include, a button, a mouse, a keyboard, a joystick, a controller, a touchscreen (e.g. of display 153), a microphone (for capturing speech or sounds from the user), a motion detector, etc.

The display 153 is a screen for sharing information to the user (e.g. temperature readings, pressure readings, a warning when a drop in temperature has been detected, etc.) The display 153 may be a screen for a computer or dongle, a touchscreen (where the display 153 may also act as a user input interface 157), etc. In some embodiments, the display 153 may be attached to the property sensors, however the display may also be an external device. When the display 135 is attached to the property sensors the display may be secured onto the skin of the patient by an adhesive. When the display is an external device, the apparatus 100 may include a transmitter for communication with the display of the external device. The transmitter may transmit signals to a receiver of an external device. After receiving a signal, the external device may display information to the user (e.g. temperature readings, pressure readings, a warning when a drop in temperature has been detected, etc.) from apparatus 100. In some embodiments an external device may display information from a plurality of apparatuses 100.

Each of, or a combination of, one or more of temperature and pressure may be used to determine a presence of limb ischemia, acute compartment syndrome and/or peripheral artery disease, detecting a change in or more of these properties for providing an indication of a presence of one or more of limb ischemia, acute compartment syndrome and peripheral artery disease.

For instance, a change in pressure over a period of time that is greater than a threshold value or that diverges from a reference value may be indicative of one or more of limb ischemia, acute compartment syndrome and peripheral artery disease, and an alert (e.g. notification, signal, etc.) may be generated for informing a user or operator of the presence of one or more of limb ischemia, acute compartment syndrome and peripheral artery disease or the detection of the change in pressure. A change in temperature over a period of time that is greater than a threshold value or that diverges from a reference value may be indicative of one or more of limb ischemia, acute compartment syndrome and peripheral artery disease, and an alert (e.g. notification, signal, etc.) may be generated for informing a user or operator of the presence of one or more of limb ischemia, acute compartment syndrome and peripheral artery disease or the detection of the change in temperature.

The apparatus 100 as described herein may therefore be used to detect, diagnose or aid in the diagnosis of one or more of limb ischemia, acute compartment syndrome and peripheral artery disease. In some instances, the apparatus 100 as described herein may therefore be used to detect, diagnose or aid in the diagnosis of limb ischemia. In some instances, the apparatus 100 as described herein may therefore be used to detect, diagnose or aid in the diagnosis of acute compartment syndrome. In some instances, the apparatus 100 as described herein may therefore be used to detect, diagnose or aid in the diagnosis of peripheral artery disease.

Even though FIG. 1A shows the apparatus 100 including a computing device 150, it will be understood that in some instances, the apparatus 100 may include as a controller, instead of the computing device 150 with a processor 151 and memory 152, a circuit board, such as when the controller only acts as a conduit to transmit the temperature readings and/or pressure readings to a remote computer (e.g. via a short-range wireless connection, such as a Bluetooth connection), such as a portable computing device (e.g. a smartphone, a smartwatch, a tablet computer).

In some embodiments, the controller of the apparatus 100 may communicate with the nearby portable computing device, which in turn may communicate the received temperature readings and/or pressure readings to a remote server. The remote server may perform the analysis of the temperature readings and/or pressure readings, to determine a state of limb ischemia by comparing the temperature readings and/or pressure readings with an expected standard (e.g. a value, an expected trend such as a change in temperature or pressure over time, an expected correlation between the temperature and the pressure over time, etc.) and generate information on the state of limb ischemia. The information on the state of limb ischemia may be transmitted to the portable computing device (e.g. for display on the portable computing device or the display 153 of the apparatus 100, the portable computing device in communication with the apparatus 100).

By the tissue or muscle of the subject, this may include a limb of the subject (a leg or an arm), or soft tissue of the subject, such as that found in the abdomen.

In some implementations, monitoring temperature and/or readings from within the limb of the patient, taken from apparatus 100, may allow the detection of a change of temperature of the limb that is indicative of a presence or change of a state of limb ischemia with respect to the limb of the patient. Apparatus 100 may be used to monitor the evolution (a change in state) of one or more pressure-related diseases (e.g. following a treatment of the disease, such as the administration of medication), such as one or more of limb ischemia, acute compartment syndrome and peripheral artery disease, to determine if the disease progresses from a reversible state of the disease to an irreversible state of the disease. Furthermore, a change of temperature observed from this monitoring may be considered a convalescence or a worsening of the pressure-related disease.

After a diagnosis, either by using apparatus 100 or other known teachings from prior art, of one or more pressure-related diseases, such as one or more of limb ischemia, acute compartment syndrome and peripheral artery disease, a treatment may be administered. Treatments for one or more pressure-related diseases, are known by one skilled in the art and may include administration of medication, endovascular treatments, surgery, etc.

After a diagnosis of one or more pressure-related diseases, such as one or more of limb ischemia, acute compartment syndrome and peripheral artery disease, the apparatus 100 may be used to monitor the evolution or change of state of the disease after an administration of a treatment. Monitoring said evolution may be indicative of the efficiency or inefficiency of the treatment.

Monitoring changes in temperature and/or pressure, or lack thereof, with apparatus 100 after treatment for one or more pressure-related diseases may lead to adjusting the treatment in accordance with the results obtained by the monitoring. Adjusting treatment as is known by one skilled in the art and may include but is not limited to increasing or decreasing medication dose; performing endovascular treatments; performing surgery or testing for other diseases. In one embodiment, a distal perfusion cannula may be inserted into a patient's limb to treat limb ischemia. Blood can flow passively through the cannula's tube, or a pump may be used to regulate and control the blood flow, ensuring optimal perfusion. In the instance that a pump is used to regulate and control blood flow, changes in temperature and/or pressure detected through use of the apparatus 100 may cause the set blood flow of the pump to change. In one instance, this change may require human intervention after the emittance of a signal from apparatus 100. In another instance, the apparatus 100 may be part of a control feedback loop, where blood flow automatically adjusts depending on the data collected by apparatus 100.

### Exemplary Software Architecture for Limb Ischemia State Evaluation:

Reference is now made to FIG. 2B, illustrating exemplary software architecture for limb ischemia state evaluation 200.

For purposes of illustration, the apparatus 100, or a remote server in communication with the apparatus 100 (e.g. via a computer that is in communication with the remote server and the apparatus 100) is described herein as having program code, stored in its memory, that includes the temperature data module 210, the standard determination module 220 and the state evaluation module 230. Each of the temperature data module 210, the standard determination module 220 and the state evaluation module 230 includes program code configured to implement the functionality of the modules as are described herein.

The temperature data module 210 includes program code stored in memory that, when executed by the processor, causes the processor to receive temperature readings generated by temperature sensor(s) implanted in limb(s) of patients. When the memory is that of a remote server, where the program code causes the processor to receive temperature readings from a plurality of temperature sensors, the temperature readings may be transmitted with metadata providing information around the received temperature readings. For instance, the metadata may include, but is not limited to, patient identification information (e.g. name of the patient, age of the patient, weight of the patient, sex of the patient, ethnicity of the patient, etc.), a time regarding when the temperature reading was taken, a unique identifier for the apparatus 100 related to the temperature sensor generating the temperature reading, a unique identifier for the temperature sensor generating the temperature reading, an identifier regarding the nature of the limb or muscle in which the temperature sensor is implanted, etc. The temperature data module 210 may cause the processor to store the received temperature readings as part of a data structure of temperature readings related to the temperature sensor that has produced the temperature readings, the patient from whom the temperature readings were taken, etc. The temperature data module 210 may also cause the processor to perform a verification protocol to confirm a permission of the apparatus 100, the remote computer and/or the temperature sensor to communicate with the server implementing the program code of the software architecture for limb ischemia state evaluation 200.

The standard determination module 220 includes program code stored in memory that, when executed by the processor, causes the processor to generate a temperature standard for purposes of comparison with the temperature readings that have been received while executing the program code of the temperature data module 210. In some embodiments, the temperature standard may be a value, such as a standard body temperature (e.g. 37 C), or a standard body temperature measured for the given patient (which may range around 37 C). In some embodiments, the temperature standard may be an expected trend or direction of temperature readings over time (e.g. a series of expected data points over time). The temperature standard may be stored in memory with metadata related to the temperature standard, such as an identifier for the patient with respect to whom the temperature standard has been determined.

The state evaluation module 230 includes program code stored in memory that, when executed by the processor, causes the processor to compare the received temperature readings to the temperature standard to perform an analysis regarding a state of limb ischemia for the muscle of the patient. For instance, when the temperature standard is a value of an expected temperature for the limb, and where the measured temperature, in accordance with the temperature readings, differentiates by a predetermined factor (e.g. 0.2-1 degrees Celsius, 1 degree Celsius, 2-3 degrees Celsius, etc.) than the expected temperature value, then a determination may be made that limb ischemia is suspected within the muscle. Moreover, the state evaluation module 230 may cause the processor to evaluate a progress of limb ischemia within the muscle by comparing the temperature readings to a temperature standard expected for a convalescence (e.g. following an administration of a treatment) of that muscle.

The state evaluation module 230 may cause the processor to generate and transmit information (e.g. a message, a report, etc.) on the state of limb ischemia within the muscle of the subject, to a remote computer, the apparatus 100, for visualization by the healthcare provider, such that the healthcare provider may respond accordingly to the received information.

In some embodiments, the state evaluation module 230 may store the information on the state of limb ischemia in a database as historical patient data, e.g. associated with metadata related to the identity of the patient and/or the identity of the temperature sensor or apparatus 100 (e.g. a unique identifier that is a serial number for that apparatus 100) that generated the temperature readings related to the information on the state of limb ischemia, in a database. In some embodiments, this stored historical patient data may be retrieved by the processor carrying out the instructions of the state evaluation module 230, to compare with contemporary temperature readings related to the patient, received from a temperature sensor, to further determine a state (e.g. a change of state—i.e. if limb ischemia is improving or worsening) of limb ischemia for that patient.

In some embodiments, the processor, carrying out the instructions of the state evaluation module 230, may determine a state of limb ischemia by comparing the temperature readings, optionally along with fields defining characteristics of the patient (e.g. sex, weight, age, height, ethnicity etc.), to a data structure of historical patient data originating from a plurality of different patients matching one or more of the fields defining the characteristics of the target patient, to determine a state of limb ischemia.

In some embodiments, the processor executing the instructions of the standard determination module 220 may also generate the temperature standard by analyzing the historical patient data of a plurality of patients, e.g., of patients with one or more fields defining the patient's characteristics that match one or more fields defining the characteristics of the target patient. For instance, if the fields of the characteristics of the target patient are defined as: “age=25”, “sex=female”, “weight=145 pounds”, height=60 inches”, a query may be generated to retrieve historical temperature data related to patients with fields matching two or more of the fields defining the characteristics of the target patient, or falling within a defined threshold range of acceptable values for each value defined in a field of the characteristics for the target patient (such as “age=between 20 and 30”; “weight=between 130 pounds and 180 pounds”; “sex=female”; “height=between 55 and 65 inches”). The processor is then caused to generate a temperature standard by analyzing the retrieved historical temperature data of the patients matching the query. In some instances, the historical data may also include a value defining a state of limb ischemia for each patient, associated to temperature data for a given period (e.g. “presence of limb ischemia”=“yes” or “no”). This value defining a state of limb ischemia for each patient may change over time, indicative of an improvement or worsening of limb ischemia. The query to retrieve historical temperature data on a plurality of patients may be refined based upon the sought state of the patient associated to the historical temperature data, e.g., if the query is to retrieve temperature data related to limb ischemia-positive patients.

In some implementations, the historical data on the patient may include pressure data, to be compared to pressure data received for a target patient. The comparison between the pressure data may provide further information on a state of a disease afflicting a muscle such as limb ischemia or compartment syndrome.

A comparison between retrieved historical data and the data for the target patient may cause a generation of a report or an indication of a suggested treatment for the target patient, determined from the comparison.

In some instances, the temperature data module 210, the standard determination module 220 and/or the state evaluation module 230 may cause the processor to query a database of electronic health records (EHR), retrieve an electronic health record corresponding to a patient relating to the received temperature readings (and in some instances, received the pressure readings), e.g., by searching for a unique identifier of the electronic health record for that patient, and cause an adjustment of the information related to the electronic health record to include information related to the received temperature readings and/or pressure readings, a temperature standard for the patient and/or information on a state of ischemia for the patient. The software architecture 200 may include or cause an interaction with an application programming interface (API) for accessing the database of electronic health records.

In some instances, the instructions carried out by the state evaluation module 230 may be executed by a processor running an artificial intelligence model. Moreover, the standard determination module 220 and/or temperature data module 210 may also include or be implemented by an artificial intelligence model for carrying out the function of the standard determination module 220 and/or temperature data module 210.

The artificial intelligence model may be one as is known in the art for performing data analysis, such as one including a convolutional neural network for deep learning. The artificial intelligence model may be provided with a dataset of historical temperature data originating from a plurality of patients, each including a value indicative of a state of limb ischemia depending on the characteristics of the patient.

A machine learning model (e.g., neural network (NN) such as a convolutional neural network or a You Only Look Once (YOLO) deep neural network architecture) can be trained by using a training dataset. Herein, the training can mean a process of determining weights and parameters of the neural network in order to determine a state of limb ischemia in a patient's muscle by analyzing the temperature data related to that patient's muscle. As a representative example of the parameter of the NN, there can be a weight given to a synapse or a bias applied to a neuron. Changing hyper-parameters and the neural network architecture can also be done during the training (convolutional neural network architecture can have multiple layers and kernels of various sizes, number of key points within a YOLO model can be varied, etc.) in order to optimize the analysis of data performed.

Examples of unsupervised learning (a subcategory of machine learning models) include but are not limited to clustering and independent component analysis.

Examples of artificial neural networks using unsupervised learning include, but are not limited to, a generative adversarial network (GAN) and an autoencoder (AE), or a convolutional neural network (CNN), etc.

Examples of supervised learning (a subcategory of machine learning models) include Supervised Vector Machine and Regression Tree.

Examples of artificial neural networks using supervised learning include convolutional neural networks (CNNs), recurrent neural networks (RNNs) and Long short-term memory (LSTM).

An exemplary deep neural network is a convolutional neural network.

In some instances, the artificial intelligence model may be trained using a dataset of historical patient data, the dataset including a plurality of data structures of temperature data, each data structure of the plurality of data structures relating to a muscle of a patient. A data structure of temperature data may include metadata with fields defining characteristics of the patient (e.g. sex, weight, age, height, ethnicity etc.), a further field indicative of a state of limb ischemia of the patient as a function of time, where the temperature data is also related to a time parameter indicative of when the temperature reading was generated. The artificial intelligence model may be caused to predict a state of limb ischemia of a tissue of a subject using a set of training temperature data. User input may be provided to the artificial intelligence model to correct the outputted determination of the state of limb ischemia of a tissue of a subject, when compared to the expected result for that set of training temperature data. Further user input may be provided until the responses generated by the artificial intelligence model fall within a tolerated margin of error (e.g. where the tolerated margin of error may be a value set by a user for the system implementing the artificial intelligence model).

### Exemplary Method of Detecting a Presence of Limb Ischemia in a Subject:

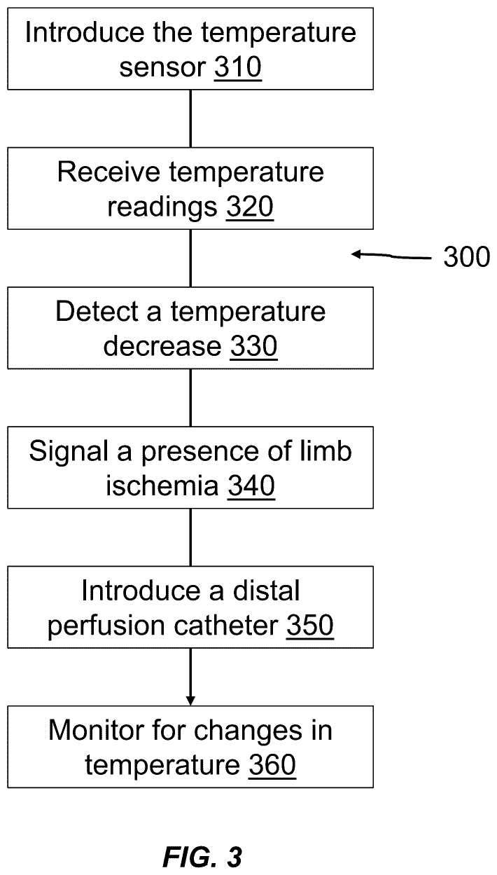

Reference is made to FIG. 3, illustrating an exemplary method 300 of detecting a presence of limb ischemia in a subject. Method 300 may be implemented using apparatus 100. However, it will be understood that method 300 may be performed using any other apparatus in accordance with the presence teachings.

The temperature sensor is introduced into the subject at step 310 at a target limb (arm, leg) (e.g. a limb with respect to which limb ischemia is suspected or is at a risk of developing). The temperature sensor is introduced into the muscle of the subject, in order to take intramuscular temperature readings of the subject. The temperature sensor is introduced into the subject using an introducer (e.g. a needle).

In some instances, a pressure sensor may also be introduced into the subject (to take intramuscular pressure readings), e.g., for detecting compartment syndrome.

Temperature readings of within the subject (within the muscle of the subject) are received over time at step 320. The temperature readings may be generated by the temperature sensor and received punctually from the temperature sensor. The temperature readings may be displayed on a display for viewing by an operator.

Similarly, when a pressure sensor has been introduced into the subject, the pressure sensor may take and transmit intramuscular pressure readings. The pressure readings may be received and displayed for viewing by an operator.

The received temperature readings are analyzed to detect a change in intramuscular temperature at step 330 (e.g. over a period of time), from a reference standard, such as a reference value (e.g. a reference value for the same limb, taken from a different and healthy limb of the subject, from a reference temperature such as 37 C, or base body temperature for that subject). For instance, a decrease in 2 or 3 Kelvin (or e.g. 2 Kelvin) from a threshold temperature value may be detected. The reference standard may also be, for instance, a value indicative of a change in temperature over time, a trend line of expected temperature values, etc.

When a change in intramuscular temperature from the threshold value is detected (e.g. in some cases, for a defined period of time), this change is indicative of a presence of limb ischemia at step 340. The computing device of the apparatus may emit information, such as a signal (e.g. a light, a vibration, a sound, a message generated on the display, electrical impulse, radio wave etc.) to inform (e.g. signal) the operator or patient that a presence of limb ischemia is suspected. Further investigation and/or treatment may follow by a medical practitioner.

In some embodiments, a change in temperature from the temperature readings may be detected (e.g. an increase, a decrease, a fluctuation). This change may indicate an improvement in the disease, a worsening of the disease and/or an indicator of the efficacy of the treatment of the disease.

Following the observed decrease in intramuscular temperature, a distal perfusion catheter or distal perfusion cannula may be inserted into the subject at step 350.

A change in intramuscular temperature may be monitored at step 360 following the introduction of the distal perfusion catheter or distal perfusion cannula. If the intramuscular temperature does not recover (e.g. an increase, a decrease) following the introduction of the distal perfusion catheter or distal perfusion cannula, the subject may be at risk for compartment syndrome.

In some instances, when a pressure sensor has been introduced into a subject, the pressure sensor may take and transmit intramuscular pressure readings. The pressure readings may be received at the computing device of the apparatus. An increase in intramuscular pressure from a threshold value may be detected (e.g. over a period of time). Following a detection of the increase in intramuscular pressure, a presence of compartment syndrome may be suspected. The apparatus may emit a signal (e.g. a light, a vibration, a sound, a message generated on the display, etc.) to signal to the operator or subject that a presence of compartment syndrome is suspected. Further investigation and/or treatment may follow, as performed by a medical practitioner.

An increase in intercompartmental pressure is a key indicator of compartment syndrome. However, a temperature change without a corresponding pressure increase suggests ischemia without compartment syndrome. In contrast, the simultaneous decline in temperature and rise in pressure indicates progressing ischemia with a high risk of developing compartment syndrome. If an intramuscular pressure is observed, the subject may be at risk for compartment syndrome, and a fasciotomy may be performed.

In some instances, monitoring temperature and pressure of a tissue over time permits detecting of a risk of reperfusion injury (if reperfusion injury is occurring, or if there is a risk of reperfusion injury in the future). Reperfusion injury may occur after perfusion of a tissue, a rapid increase of blood flow to the tissue (e.g. limb) that has been underperfused, e.g. due to an occlusion to an artery or vein, once the occlusion has been removed from the artery or vein that had been occluded, where the occluded artery or vein was the cause of limb ischemia.

The following exemplary study is provided to enable the skilled person to better understand the present disclosure. As it is solely illustrative and representative examples, they should not limit the scope of the present disclosure, only added for illustrative and representative purposes. It will be understood that other exemplary studies may be used to further illustrate and represent the present disclosure without departing from the present teachings.

### Exemplary Study:

Temperature sensors and pressure sensors were introduced into the limbs of each of ten subjects.

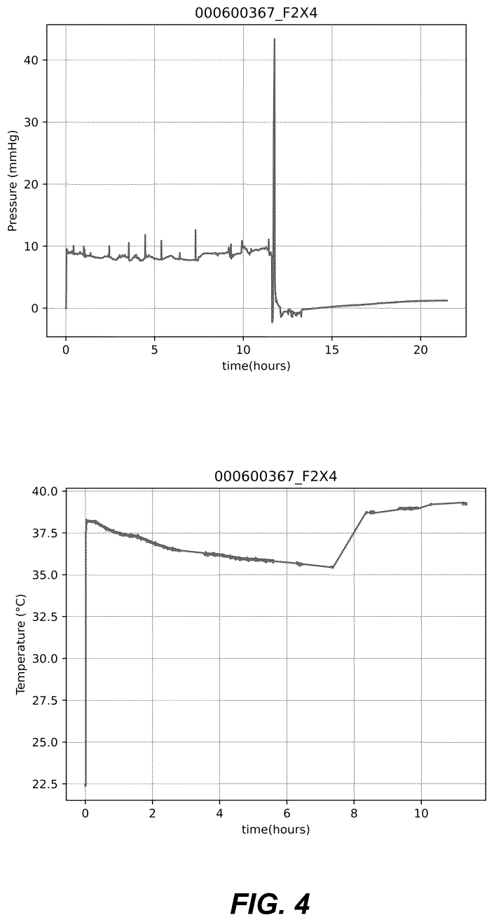

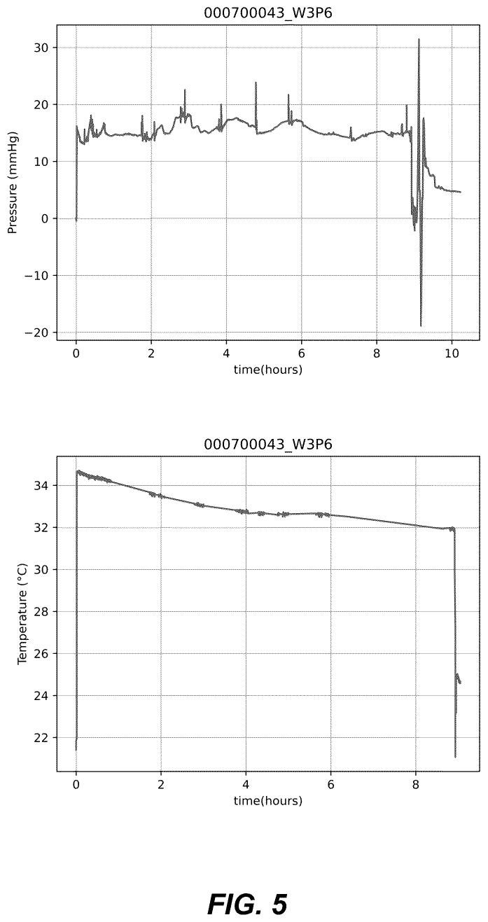

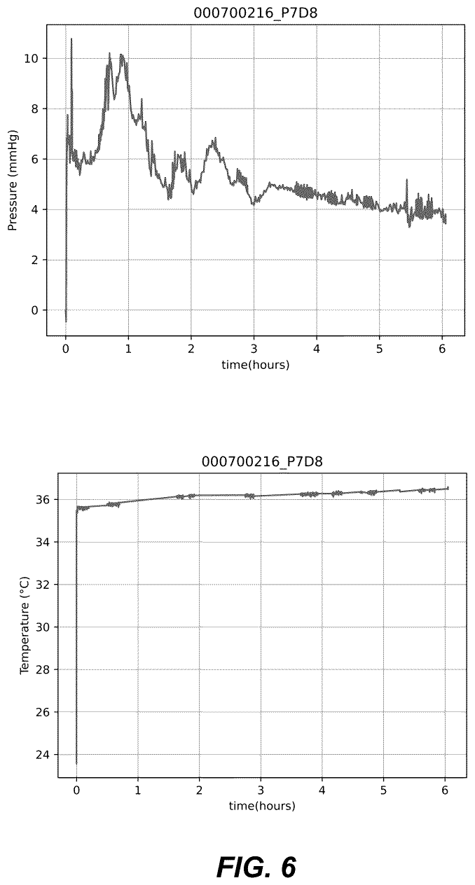

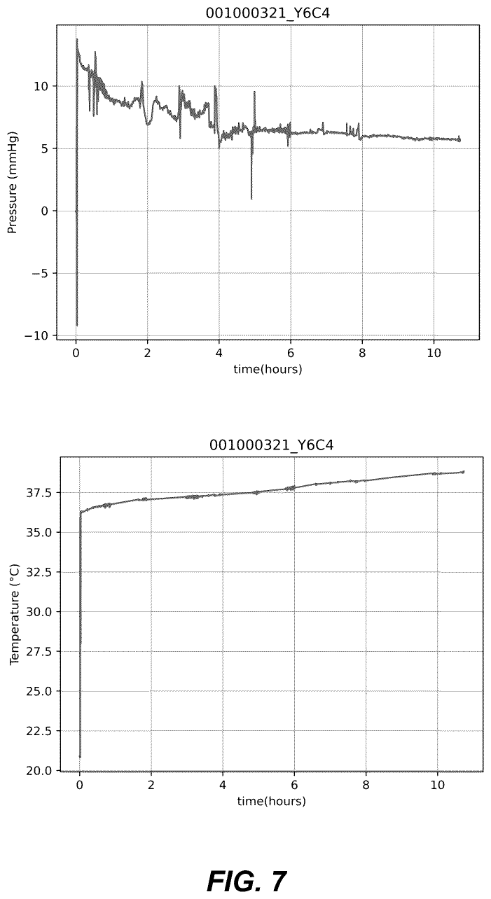

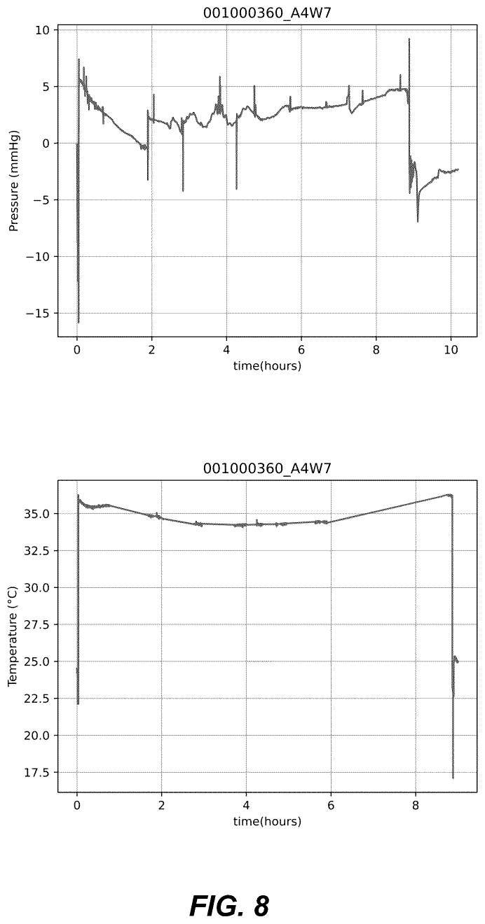

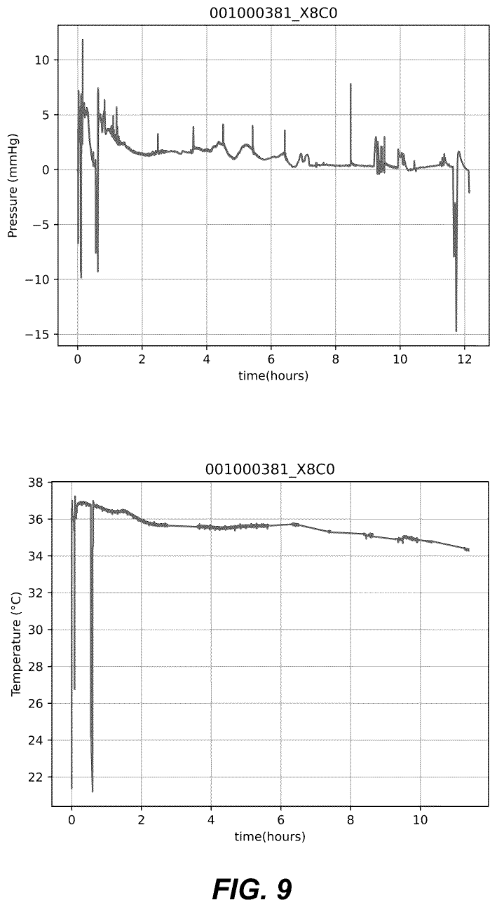

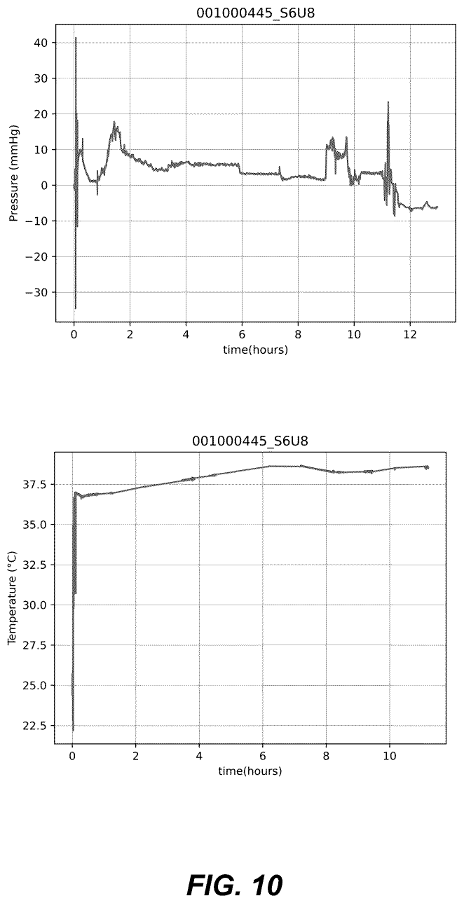

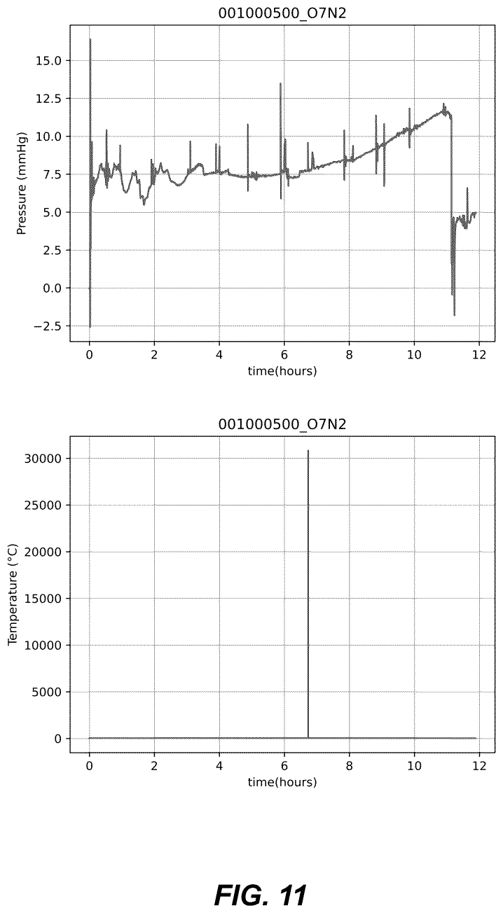

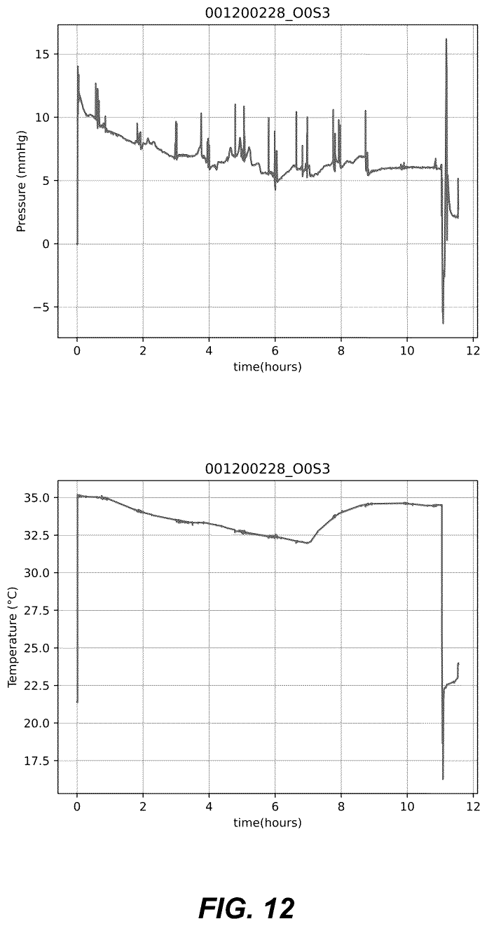

Intramuscular pressure and intramuscular temperature were monitored over time for each subject. The pressure readings and the temperature readings are presented in the graphs of FIGS. 4-12. In some instances, a temperature within a muscle may drop by 9 degrees Celsius. Temperature changes to as low as 28° C. have been observed, with a noted correlation between temperature and pressure, suggesting an interdependent relationship between these physiological parameters.

As such, it has been discovered that a variation in intramuscular temperature, when optionally analyzed alongside intramuscular pressure, may provide information on a state of ischemia for the muscle.

A decrease in intramuscular temperature was observed prior to an increase in intramuscular pressure. As such, a decrease in intramuscular temperature may be indicative of a presence of limb ischemia, and a risk for compartment syndrome if intramuscular pressure is maintained at a level above a reference value or continues to increase.

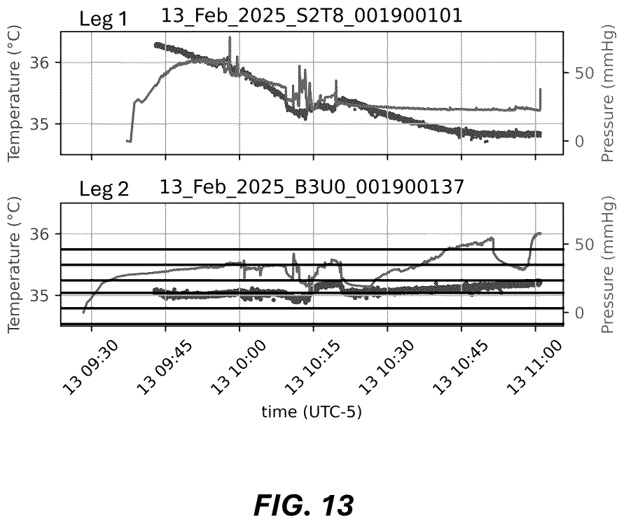

FIG. 13 illustrates temperature readings and pressure readings over time of both legs of a patient underdoing a procedure using a distal perfusion cannula inserted in each leg. As can be observed by a change in temperature and pressure over time for each leg, the variations in temperature over time follow closely the corresponding change in pressure, where a change in pressure is followed by a corresponding variation in temperature. Variations as low as 0.2° C. in intramuscular temperature can be observed following changes in intramuscular pressure. Variations in intramuscular temperature can be increasing or decreasing intramuscular temperatures. Variations in intramuscular temperature may also produce trends correlated with variations in intramuscular pressure such that the variations in intramuscular temperature may be indicative of a presence, absence or development of limb ischemia and/or compartment syndrome. In another instance, the variations in intramuscular temperature may predict a presence, development or remediation of limb ischemia and/or compartment syndrome. As such, monitoring the change in pressure over time, and the corresponding change in temperature, for a given tissue, may provide insight on the overall health of the limb (and if compartment syndrome or limb ischemia is suspected). Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

What is claimed is:

1. An apparatus for use in monitoring a state of or detecting a presence of limb ischemia within a limb of a subject, comprising:
a temperature sensor configured to obtain intramuscular temperature readings when implanted using an introducer into the limb;
a controller that is configured to:
receive the temperature readings from the temperature sensor; and
at least one of:
display the temperature readings on a display; and
analyze the temperature readings to identify a change in intramuscular temperature when compared to a reference temperature value, and following the identifying, generate information indicative of the change of intramuscular temperature,
whereby the change in intramuscular temperature is indicative of a change in the state of limb ischemia or the presence of limb ischemia.

2. The apparatus as defined in claim 1, further comprising the introducer.

3. The apparatus as defined in claim 1, further comprising the display, and wherein the controller is configured to display the temperature readings on the display.

4. The apparatus as defined in claim 1, wherein the controller is configured to analyze the temperature readings to identify a change in intramuscular temperature when compared to a reference temperature value, and following the identifying, generate the information indicative of the change of intramuscular temperature.

5. The apparatus as defined in claim 4, wherein the information is a signal, and the signal is generated following a determination that the identifying the change in intramuscular temperature is occurring over a set period of time according to a period of time value.

6. The apparatus defined in claim 4, wherein the change in intramuscular temperature is a decrease in intramuscular temperature for detecting the presence of limb ischemia.

7. The apparatus as defined in claim 4, wherein the information is a signal that is a message appearing on a display.

8. The apparatus as defined in claim 1, further comprising a pressure sensor for insertion into the limb, wherein the controller is further configured to receive pressure readings generated by the pressure sensor.

9. The apparatus as defined in claim 8, wherein the controller is further configured to perform at least one of:
display the pressure readings on the display; and
analyze the pressure readings to identify a change in intramuscular pressure when compared to a reference pressure value, and following the identifying, generate a signal indicative of the change of intramuscular pressure,
whereby the change in intramuscular pressure provides information on a presence of compartment syndrome within the limb.

10. The apparatus as defined in claim 1, wherein the temperature sensor is adapted to transmit the temperature readings to the controller via a wired connection.

11. The apparatus as defined in claim 1, further comprising a user input interface for receiving user input.

12. The apparatus as defined in claim 1, wherein the temperature sensor is a first temperature sensor, further comprising a plurality of temperature sensors including the temperature sensor, and wherein the controller is further configured to receive temperature readings from the plurality of temperature sensors.

13. A method of detecting a presence of limb ischemia within a limb of a subject, comprising:
receiving intramuscular temperature readings of the limb received from a temperature sensor implanted in the limb;
analyzing the temperature readings to identify a decrease in intramuscular temperature within the limb when compared to an expected temperature standard; and
outputting information indicative of the decrease in intramuscular temperature, that is indicative of a presence or a state of limb ischemia within the limb of the subject.

14. The method as defined in claim 13, further comprising introducing a distal perfusion cannula within the limb.

15. The method as defined in claim 14, further comprising monitoring intramuscular temperature following the introduction of the distal perfusion cannula to detect an increase in intramuscular temperature.

16. The method as defined in claim 15, further comprising, following an absence of the increase in intramuscular temperature, generating an alert indicative of a risk of compartment syndrome and low perfusion that may be tied to tissue necrosis or acute coronary syndrome.

17. The method as defined in claim 16, wherein the reference intramuscular temperature value is an intramuscular temperature value of a healthy limb of the subject.

18. The method as defined in claim 16, wherein the signal is generated using a signal transducer.

19. The method as defined in claim 16, further comprising implanting the temperature sensor into the limb using an injector.

20. The method as defined in claim 16, further comprising:
receiving pressure readings of the limb received from a pressure sensor implanted in the limb;
analyzing the pressure readings to identify a change in pressure within the limb when compared to a reference value; and
outputting information indicative of the change in pressure, indicative of an abnormality within the limb of the subject.
